SEQUENCE LISTING (1 sequence)

SEQ ID NO: 1 - protein, 31 aa, Homo sapiens
  Amino Acid sequence of Liraglutide
HAEGTFTSDVSSYLEGQAAKEFIAWLVRGRG

(19) United States
(12) Patent Application Publication
(10) Pub. No.: US 2017/0283478 A1
(43) Pub. Date: Oct. 5, 2017

(54) PROCESS FOR PREPARATION OF LIRAGLUTIDE

(71) Applicant: DR. REDDY'S LABORATORIES LIMITED, Hyderabad (IN)

(72) Inventors: Sunil Kumar GANDAVADI, Hyderabad (IN); Sekhar NARIYAM MUNASWAMY, Hyderabad (IN); Karthik RAMASAMY, Hyderabad (IN); Rajiv Vishnukant THAKUR, Bhavnagar (IN); Yagna Kiran Kumar KOMARAVOLU, Hyderabad (IN); Kishore Kumar MOVVA, Vuyyuru (IN); Giri Babu NANDIVADA, Srikakulam (IN); Lavanya KOLA, Chittor (IN); Srinivas KATKAM, Hyderabad (IN)

(21) Appl. No.: 15/325,119

(22) PCT Filed: Jul. 13, 2015
(86) PCT No.: PCT/IB2015/055273

§ 371 Date: Jan. 10, 2017

(30) Foreign Application Priority Data
Jul. 11, 2014 (IN) 3453/CHE/2014

(51) Int. Cl. C07K 14/605

(52) U.S. Cl. CPC C07K 14/605

(57) ABSTRACT

The present application relates to improved processes for the preparation of Liraglutide.

### INTRODUCTION

Aspects of the present application relates to improved processes for the preparation of Liraglutide.

Liraglutide, marketed under the brand name Victoza, is a long-acting glucagon like peptide agonist developed by Novo Nordisk for the treatment of type 2 diabetes.

Liraglutide is an injectable drug that reduces the level of sugar (glucose) in the blood. It is used for treating type 2 diabetes and is similar to exenatide (Byetta). Liraglutide belongs to a class of drugs called incretin mimetics because these drugs mimic the effects of incretins. Incretins, such as human-glucagon-like peptide-1 (GLP-1), are hormones that are produced and released into the blood by the intestine in response to food. GLP-1 increases the secretion of insulin from the pancreas, slows absorption of glucose from the gut, and reduces the action of glucagon. (Glucagon is a hormone that increases glucose production by the liver.) All three of these actions reduce levels of glucose in the blood. In addition, GLP-1 reduces appetite. Liraglutide is a synthetic (man-made) hormone that resembles and acts like GLP-1. In studies, Liraglutide treated patients achieved lower blood glucose levels and experienced weight loss.

Liraglutide, an analog of human GLP-1 acts as a GLP-1 receptor agonist. The peptide precursor of Liraglutide, produced by a process that includes expression of recombinant DNA in Saccharomyces cerevisiae, has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of Liraglutide is C₁₇₂H₂₆₅N₄₃O₅₁ and the molecular weight is 3751.2 Daltons. It is represented by the structure of formula (I)

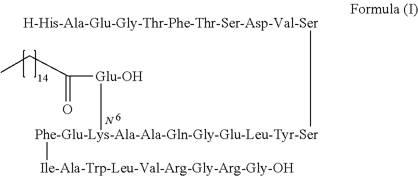

U.S. Pat. No. 7,572,884 discloses a process for preparing Liraglutide by recombinant technology followed by acylation and removal of N-terminal extension.

U.S. Pat. Nos. 7,273,921 and 6,451,974 discloses a process for acylation of Arg-³⁴GLP-1(7-37) to obtain Liraglutide.

U.S. Pat. No. 8,445,433 discloses a solid phase synthesis of Liraglutide using a fragment approach.

International Application publication No. WO2013037266A1 discloses solid phase synthesis of Liraglutide, characterized in that comprises A) the presence of the activator system, solid phase carrier and by resin Fmoc protection N end obtained by coupling of glycine (Fmoc-Gly-OH) Fmoc-Gly-resin; B) by solid phase synthesis, prepared in accordance with the sequentially advantage Liraglutide principal chain N end of the coupling with Fmoc protected amino acid side chain protection and, wherein the lysine using Fmoc-Lys (Alloc)-OH; C) Alloc getting rid of the lysine side chain protecting group; D) by solid phase synthesis, the lysine side chain coupling Palmitoyl-Glu-OtBu; E) cracking, get rid of protecting group and resin to obtain crude Liraglutide; F) purification, freeze-dried, to obtain Liraglutide.

Even though, the above mentioned prior art discloses diverse processes for the preparation of Liraglutide, they are often not amenable on commercial scale because of expensive amino acid derivatives such as pseudo prolines used in those processes.

Hence, there remains a need to provide simple, cost effective, scalable and robust processes for the preparation of Liraglutide involving commercially viable amino acid derivatives and reagents.

### SUMMARY

In the first embodiment, the present invention relates to a process for the preparation of Liraglutide of formula (I), which includes one or more of the following steps:
a) introducing the spacer N^{α}-Palmitoyl-L-γ-glutamyl-OtBu on side chain NH₂ of Lysine of fragment Fmoc-Lys(Alloc)-Glu(Otbu)-Phe-Ile-Ala-Trp(Boc)-Leu-Val-Arg(Pbf)-Gly-Arg(pbf)-Gly attached to solid support;
b) coupling rest of the amino acid sequence from alpha amino group of Lysine of the fragment of Liraglutide obtained in step (a) to obtain Liraglutide attached to solid support;
c) cleavage of Liraglutide from the solid support;
d) optionally purifying the resulting Liraglutide.

In the second embodiment, the present invention relates to a process for the preparation of Liraglutide of formula (I), which includes one or more of the following steps:
a) sequential coupling of the fragments Fmoc-Arg(pbf)-Gly-OH, Fmoc-Leu-Ala-Arg(Pbf)-OH, Fmoc-Ile-Ala-Trp(Boc)-OH, Fmoc-Glu(OtBu)-Phe-OH, Fmoc-Glu(OtBu)-Phe-OH, Fmoc-Lys-(Glu-(Palmitic Acid)-OH, Fmoc-Gly-Gln(Trt)-Ala-Ala-OH, Fmoc-Tyr(tBu)-Leu-Glu(OtBu)-OH, Fmoc-Val-SerSer(tBu)-Ser(tBu)-OH, Fmoc-Phe-Thr(tBu)-Ser(tBu)-Asp(tBu)-OH, Fmoc-Gly-Thr(tBu)-OH and Boc-His(Trt)-Ala-Glu(OtBu)-OH on Glycine that is already attached to solid support to obtain Liraglutide attached to solid support;
b) cleavage of Liraglutide from the solid support;
c) optionally purifying the resulting Liraglutide.

### DETAILED DESCRIPTION

The present invention provides processes for the preparation of Liraglutide of formula (I).

In the first embodiment, the present invention relates to process for the preparation of Liraglutide of formula (I), which includes one or more of the following steps:
a) introducing the spacer N^{α}-Palmitoyl-L-γ-glutamyl-OtBu on side chain NH₂ of Lysine of fragment Fmoc-Lys(Alloc)-Glu(Otbu)-Phe-Ile-Ala-Trp(Boc)-Leu-Val-Arg(Pbf)-Gly-Arg(pbf)-Gly attached to solid support;
b) coupling rest of the amino acid sequence from alpha amino of Lysine of the fragment of Liraglutide obtained in step (a) to get Liraglutide;
c) cleavage of Liraglutide from the solid support;
d) optionally purifying the resulting Liraglutide.

Step (a) of the first embodiment involves introducing the spacer N^{α}-Palmitoyl-L-γ-glutamyl-OtBu on side chain NH₂ of Lysine of fragment Fmoc-Lys(Alloc)-Glu(Otbu)-Phe-Ile-Ala-Trp(Boc)-Leu-Val-Arg(Pbf)-Gly-Arg(pbf)-Gly attached to solid support.

The fragment Fmoc-Lys(Alloc)-Glu(Otbu)-Phe-Ile-Ala-Trp(Boc)-Leu-Val-Arg(Pbf)-Gly-Arg(pbf)-Gly attached to solid support can be prepared by the processes well known in the art. For example, Gly is attached to solid support first and then rest of the amino acids of the above fragment are coupled sequentially in that order. Alloc on the fragment Fmoc-Lys(Alloc)-Glu(Otbu)-Phe-Ile-Ala-Trp(Boc)-Leu-Val-Arg(Pbf)-Gly-Arg(pbf)-Gly can be removed by the processes well known in the art, preferably using palladium tetrakis and phenyl silane.

Nα-Palmitoyl-L-γ-glutamyl-OtBu is coupled to the fragment Fmoc-Lys-Glu(Otbu)-Phe-Ile-Ala-Trp(Boc)-Leu-Val-Arg(Pbf)-Gly-Arg(pbf)-Gly on the solid support in the presence of coupling reagents well known in the art.

Step (b) of the present invention involves coupling rest of the amino acid sequence from alpha amino of Lysine of the fragment of Liraglutide obtained in step (a) to get Liraglutide. Coupling of the amino acid sequence from alpha amino of Lysine of the fragment of Liraglutide obtained in step (a) can be prepared by the processes well known in the art.

The solid support used for the preparation of Liraglutide fragment Lys(Alloc)-Glu(Otbu)-Phe-Ile-Ala-Trp(Boc)-Leu-Val-Arg(Pbf)-Gly-Arg(pbf)-Gly include resin. Resin is a solid, non-soluble support material, controlled pore size glass, silica or more commonly a polymeric organic resin such as for instance the classical polystyrene-divinylbenzene resin (PS resin) used by Merrifield along with hydroxybenzyl-phenyl integral linker moieties for attaching peptide thereto or PS resin used by Wang with hydroxy-benzyl-p-benzyloxy moieties directly linked to the resin. Resins as used in the present invention are of standard mesh size (US bureau of standards), which is about 50-500 mesh, more preferably 100 to 400 mesh.

The resin is an acid-labile solid support which liberates a C-terminal carboxylic acid upon cleavage of the peptide from the solid support. Examples of such resins include but not limited to 2′-chloro-trityl, 4-methoxy or 4,4′-dimethoxy-trityl, 4-methyltrityl resins, 2-(4-hydroxy-phenyl)-2,2-diphenyl-acetyl resin, Rink acid resin (4-(2′,4′-dimethoxyphenyl-hydroxymethyl)phenoxy, HMPB-resin, hypogel resin, tentagel resin, etc.,

Coupling reagents for peptide synthesis are well-known in the art (see Bodansky, M., Principles of Peptide Synthesis, 2^{nd} ed. Springer Verlag Berlin/Heidelberg, 1993). Coupling reagents may be mixed anhydrides (e.g. T3P: propane phosphonic acid anhydride) or other acylating agents such as activated esters or acid halogenides (e.g. ICBF, isobutyl-chloroformate), or they may be carbodiimides (e.g. 1-ethyl-3-(3-dimethylaminopropyl)-carbodiimide, diisopropyl-carbodiimide), activated benzotriazine-derivatives (DEPBT: 3-(diethoxyphosphoryloxy)-1,2,3-benzotriazine-4(3H)-one) or uronium or phosphonium salt derivatives of benzotriazol.

In view of best yield, short reaction time and protection against racemization during chain elongation, more preferred is that the coupling reagent is selected from the group consisting of uronium salts and phosphonium salts of the benzotriazol capable of activating a free carboxylic acid function along with that the reaction is carried out in the presence of a base. Suitable and likewise preferred examples of such uronium or phosphonium coupling salts are e.g. HBTU, BOP, PyBOP, PyAOP, HCTU, TCTU, HATU, TATU, TOTU, HAPyU, etc.,

The amount of individual coupling agents used may range from about 1 to about 6 molar equivalents, per molar equivalent of resin with respect to resin loading capacity. Preferably, 3 molar equivalents of individual coupling agents per molar equivalent of the resin with respect to resin loading capacity may be used.

Deprotection of the base labile may be carried out as routinely done in the art, e.g. with 20% piperidine in N-methyl pyrrolidone (NMP), dichloromethane (DCM) or dimethylformamide (DMF). Both organic apolar aprotic solvents are routinely applied in the art for all steps of solid-phase synthesis. NMP or DMF is a preferred solvent.

Fmoc amino acids, dipeptides, tripeptides or oligopeptides are preferably coupled with normal 1-3 eq., more preferably with only 1-2 eq. of such Fmoc amino acid reagent per eq. of reactive, solid-support bound amino function as determinable e.g. by Kaiser test. The coupling temperature is usually in the range of from 15 to 30° C., especially where using phosphonium or uronium type coupling reagents. Typically, a temperature of about 20 to 25° C. is applied for coupling. It is an advantage of the method of the present invention having devised a method of synthesis allowing high yield of product or excellent purity of GLP-1 product without being forced to use precious, bio hazardous reagents in excessive amount, essentially wasting most of that excess in the reaction's effluents.

Further, the following alternative protecting groups or protective groups well known in the art can be used to protect amino acids or their derivatives, which are used in the process of the present invention. Commonly employed carboxy-protection groups for Glu, Asp are e.g. Mpe, O-1-Adamantyl, O-benzyl and even simply alkyl esters may be used, though less commonly used. For sake of ease, typically and preferably tert.butyl groups are used. Tyrosine may be protected by different protection groups, e.g. tert.butyl ether or Z- or more preferably 2-Bromo-Z esters. It is equally possible to use tritylalcohol protection groups such as 2-chloro-trityl or 4-methoxy or 4, 4′ methoxy-trityl groups. Preferably, it is a trityl or a tert.butyl protection group. More preferably, it is a tertiary butyl (tBu) protection group, meaning the tyrosyl side chain is modified to tertiary-butyl ether. The tBu group is only efficiently removed under strongly acidic condition. Arginine protection group may be preferably selected from the group consisting of 2,2,4,6,7-pentamethyldihydrobenzofuranyl-5-sulfonyl (Pbf), adamantyloxy-carbonyl and isobornyl-oxy-carbonyl, 2,2,5,7,8-pentamethylenchromanesulfonyl-6-sulfonyl (Pmc), 4-methoxy-2,3,6-trimethylbenzenesulfonyl (Mtr) and its 4-tert.butyl-2,3,5,6-tetramethyl homologue (Tart) or Boc, which are only cleaved under strongly acidic conditions as defined above. More preferably, it is Pbf, Pmc, Mtr, most preferably, it is Pbf; upon global deprotection of side chains under strongly acidic conditions, in usually aqueous medium, bystander-alkylation of deprotected tyrosine is not observed with Pmc,Mtr and esp. Pbf. Pbf's cleavage rate is the highest ever. Ser, Thr typically may be typically and preferably protected by e.g. tert.-butyl or trityl, most preferably tert-butyl. Other modes of protection are equally feasible, e.g. with benzyl, though less preferred since eventually requiring hydrogenolytic removal or extended incubation at strongly acidic incubation, which is both equally undesirable. Similar considerations apply to protection of Lys; typically and preferably, Lys is protected with Boc, Alloc, Mtt, ivDde, TCP. Trp must not necessarily be protected during solid-phase synthesis, though protection with typically Boc is preferred.

The functional group present on the amino acids used in the process of the present invention may be appropriately protected to avoid any undesired side reaction products. Suitable protective groups are described in the Literature (see, for example, P Wuts and T. W. Greene, “Protective Groups in Organic Synthesis”, John Wiley & Sons, 4^{th} edition, 2007). The protecting group may vary depending upon the particular amino acid which may include, but are not limited to Boc, Pbf, tBu and Trt.

Optionally, the coupling of amino acid with preferred molar equivalents may also be carried out in two steps to increase the coupling efficiency, wherein the coupling reagent or protected amino acid or both may be utilized in two or more lots.

The coupling reaction may be carried out in a suitable solvent. The term “suitable solvent” refers to any solvent, or mixture of solvents, that afford a medium within which the desired reaction is carried out. The solvents that may be used in the coupling step include but are not limited to dichloromethane, tetrahydrofuran, dimethylformamide, N-methylpyrrolidone or the mixtures thereof.

After the completion of the reaction, the resin may be optionally washed with solvents such as dichloromethane, dimethylformamide to remove residual reagents and byproducts. The process may be repeated, if desired.

The coupling efficiency after each coupling step may be monitored during synthesis by means of a Kaiser test or any other suitable test (HPLC). The individual coupling steps, if showing unexpectedly low coupling efficiency may also be repeated prior to proceeding for deprotection and coupling with next amino acid of the sequence.

The Fmoc protected amino acids are commercially available or may be prepared according to procedures known in the art.

Step (c) of the present invention involves cleavage of Liraglutide from the solid support.

The cleavage of the peptide from the solid support may be accomplished by any conventional methods well known in the art. The process results in cleaving the peptide from the solid support and global deprotection of the side chain protecting groups of the amino acids to provide Liraglutide. The overall process may be carried out in an inert atmosphere, i.e. Nitrogen or Argon.

The step of cleaving the peptide from the resin involves treating the protected peptide anchored to the resin with an acid and at least one scavenger. The peptide cleavage reagent used in the process of the present invention is a cocktail mixture of acid, scavengers and solvents.

The acid utilized in the cleavage reagents may be selected from trifluoroacetic acid (TFA), difluoroacetic acid or monofluoroacetic acid. Scavengers such as EDT, DDM, TES, TIS, phenol, thioanisole and water or in any combination thereof may be used in the process of the present invention.

Preferably, a cocktail mixture comprising TFA/Phenol/Thioanisole/Water/Triisopropyplsilane (TIS) in a ratio of about 82.5%, 5%, 5%, 2.5%, and 5% respectively may be used as the peptide cleaving and global deprotecting reagent. More preferably, a cocktail mixture comprising TFA/Phenol/Water/TIS in a ratio of about 76.5%, 17.5%, 4.3%, and 1.7% respectively may also be used as the peptide cleaving and global deprotecting reagent.

The solvent used in this cleaving step of the process of the present invention may be selected from but not limited to dichloromethane, trichloromethane or the like. Dichloromethane is used as preferred solvent. The solvent may also help in swelling the resin prior to effective cleavage of the peptide.

The temperature at which the cleavage and global deprotection may be carried out ranging from about 15° C. to about 40° C. Preferred temperature for cleavage and global deprotection may be in the range of about 25°−30° C.

After the completion of the reaction, the reaction mixture may optionally be filtered and washed with acid or an organic solvent. Crude Liraglutide may be isolated by combining the reaction mass with an organic solvent, preferably by combining with an ether solvent. Ether solvents that may be used include but are not limited to diethyl ether, diisopropyl ether, tert-butyl methyl ether, tert-butyl ethyl ether, tert-amyl methyl ether, isopropyl ether and the like or combinations thereof.

The isolation may be carried out by adding an ether solvent to the reaction mass or by adding reaction mass to the ether solvent selected. Preferably, the reaction mass is added to an ether solvent. More preferably, the reaction mass is added to an ether solvent precooled to a temperature of about −5° C. to about 5° C.

The obtained suspension may be maintained at a temperature of about 0° C. to about 15° C., preferably at a temperature of about 0° C. to about 5° C. to effect the complete precipitation of the product. The obtained precipitate may be separated using conventional techniques known in the art. One skilled in the art may appreciate that there are many ways to separate a solid from the mixture, for example it can be separated by using techniques such as filtration by gravity or by suction, centrifugation, decantation, and the like. The obtained crude product may be optionally washed with an organic solvents preferably ether and subjected to drying under continuous nitrogen purging.

Step (d) of the present invention involves optionally purifying the resulting Liraglutide.

The purification process of Liraglutide can be carried out by the processes well known in art. Purification process include but not limited to preparative reverse phase HPLC, ion exchange chromatography, size exclusion chromatograph, affinity chromatography, etc.,

The purification process of Liraglutide is carried out on preparative reverse phase HPLC, wherein often a C-18 or C-8 column is utilized on reversed phase Purification for crude Liraglutide is carried out firstly in a neutral gradient medium, which comprises dissolution of crude peptide in tris buffer and loading onto the column. The material is then eluted with a gradient of tris buffer acetonitrile on a column.

Preferably the gradient of tris buffer may have concentration of about 10 mM and acetonitrile (Buffer B) volumes may range from 60-40/40-60. During elution, fractions are collected at regular intervals using a preparative HPLC system. The collected fractions are assayed by HPLC to determine the purity and fraction with desired purities may be pooled together for further purification.

The pooled fraction obtained from the previous purification step can be subjected to further purification by elution with a gradient comprising of Trifluoroacetic acid and Acetonitrile. The concentration of trifluoroacetic acid may be about 0.05% to about 1% in water. During the elution; the desired pure fractions are collected again and assayed by HPLC. The purified Liraglutide pooled fractions are then subjected to desolvation to remove acetonitrile solvent. Fractions with desired purity preferably greater than 96.5% purity may be considered as pure fractions.

The pure pooled fraction so obtained may be subjected to lyophilization under the set parameters of lyophilization to collect the lyophilized powder which may be assayed by purity method of HPLC to ensure that it meets API specifications.

In the second embodiment, the present invention relates to a process for the preparation of Liraglutide of formula (I), which includes one or more of the following steps:
a) sequential coupling of the fragments Fmoc-Arg(pbf)-Gly-OH, Fmoc-Leu-Ala-Arg(Pbf)-OH, Fmoc-Ile-Ala-Trp(Boc)-OH, Fmoc-Glu(OtBu)-Phe-OH, Fmoc-Glu(OtBu)-Phe-OH, Fmoc-Lys-(Glu-(Palmitic Acid)-OH, Fmoc-Gly-Gln(Trt)-Ala-Ala-OH, Fmoc-Tyr(tBu)-Leu-Glu(OtBu)-OH, Fmoc-Val-SerSer(tBu)-Ser(tBu)-OH, Fmoc-Phe-Thr(tBu)-Ser(tBu)-Asp(tBu)-OH, Fmoc-Gly-Thr(tBu)-OH and Boc-His(Trt)-Ala-Glu(OtBu)-OH on Glycine attached to solid support to get Liraglutide;
b) cleavage of Liraglutide from the solid support;
c) optionally purifying the resulting Liraglutide.

Step (a) of the present invention involves sequential coupling of the fragments Fmoc-Arg(pbf)-Gly-OH, Fmoc-Leu-Ala-Arg(Pbf)-OH, Fmoc-Ile-Ala-Trp(Boc)-OH, Fmoc-Glu(OtBu)-Phe-OH, Fmoc-Glu(OtBu)-Phe-OH, Fmoc-Lys-(Glu-(Palmitic Acid)-OH, Fmoc-Gly-Gln(Trt)-Ala-Ala-OH, Fmoc-Tyr(tBu)-Leu-Glu(OtBu)-OH, Fmoc-Val-SerSer(tBu)-Ser(tBu)-OH, Fmoc-Phe-Thr(tBu)-Ser(tBu)-Asp(tBu)-OH, Fmoc-Gly-Thr(tBu)-OH and Boc-His(Trt)-Ala-Glu(OtBu)-OH on Glycine attached to solid support to get Liraglutide. Sequential coupling of fragments on the solid support, cleavage of the peptide from the solid support and purifying the crude peptide can be performed by the processes well known in the art as described above in the case of first embodiment.

An advantage of the present process is in the introduction of spacer N^{α}-Palmitoyl-L-γ-glutamyl-OtBu on side chain NH₂ of Lysine at the 12^{th} position from C terminal of Liraglutide. In contrast to the reported process wherein the acylation is carried out after the principle chain of Liraglutide is obtained, in which case there is a possibility of formation of diacylated or triacylated peptide impurities. The possibilities of diacylated or triacylated peptide impurities can be resolved by using the process of the present invention.

### Definitions

The following definitions can be used in connection with the words or phrases used in the present application unless the context indicates otherwise.

The term “amino acid” as used herein refers to an organic compound comprising at least one amino group and at least one carboxylic acid group. The amino acid may be a naturally occurring amino acid or be of synthetic origin, or an amino acid derivative or amino acid analog.

The term “protected amino acids” or “amino acid derivative” as used herein, refers to amino acids where functional groups in amino acids are derivatized with a suitable protecting group.

The term “protecting group” as used herein, refers to a group used to protect a certain functional group for preventing it from participating in a chemical reaction and after the completion of said chemical reaction, the said protecting group should be removed from the said functional group easily.

The abbreviations used in the present description are defined below
Alloc—Allyloxy carbonyl
Boc—tertiary-Butyloxycarbonyl
BOP—Benzotriazol-1-yl-oxy-tris-(dimethylamino)-phosphonium hexafluorophosphate
DBU—1,8-Diazobicycol[5.4.0]undec-7-ene
DCC—N,N-Dicyclohexylcarbodiimide
DIC—N,N-Diisopropylcarbodiimide
DCM—Dichloromethane
DMF—N,N-Dimethylformamide
EDT—1,2-Ethanedithiol
EDTA—Ethylenediamine tetra acetic acid
EEDQ—2-Ethoxy-1-ethoxycarbonyl-1,2-di hydroquinoline
DDM—Dodecane mercaptan
iv Dde—4,4-dimethyl-2,6-dioxocyclohex-1-ylidene)-3-methylbutyl
IIDQ—Isobutyl 1,2-dihydro-2-isobutoxy-1-quinolinecarboxylate
Fmoc—9-Fluorenylmethyloxycarbonyl
HBTU—2-(1H-Benzotriazol-1-yl)-1,1,3,3-tetramethyluronium hexafluorophosphate
HOBt—N-hydroxybenzotriazole
Mtt—Methyl trityl
Pbf—2,2,4,6,7-Pentamethyldihydrobenzofuran-5-sulfonyl
PyBOP—Benzotriazol-1-yl-oxy-tris-(pyrrolidino)-phosphonium hexafluorophosphate
TBTU—O-Benzotriazol-1-yl-1,1,3,3-tetramethyluronium tetrafluoroborate
tBu—Tertiary butyl
TCP—Tetral chloro phthalyl
TFA—Trifluoroacetic acid
TES—Triethylsilane
TIS—Triisopropylsilane
Trt—Trityl
HMPB—4-hydroxymethyl-3-methoxyphenoxybutyryl

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

### Example 1

Stage I: Preparation of Wang Resin-Gly-Arg(pbf)-Gly-Arg(pbf)-Val-Leu-Trp(Boc)-Ala-Ileu-Phe-Glu(Otbu)-Lys-{Glu(OH)-NH(palmitoyl)}-Ala-Ala-Gln(trt)-Gly-OH-Glu(Otbu)-Leu-Tyr(Otbu)-Ser(Otbu)-Ser(Otbu)-Val-Asp(Otbu)-Ser(Otbu)-Thr(Otbu)-Phe-Thr(Otbu)-Gly-Glu(Otbu)-Ala-Boc-His(trt)-OH.

Wang resin (50 gm) is swelled in DCM (500 ml) for 1 hr in a sintered flask. DCM was filtered using Vacuum. Fmoc-Glycine (44.6 gm, 150 mmol) was dissolved in dichloromethane (250 ml). 1-(2-mesitylene sulfonyl)-3-nitro-1H-1,2,4 triazole (44.4 gm, 150 mmol) and 1-methyl imidazole (9 ml, 112 mmol) was then added. The reaction mixture was added to wang resin and stirred for 3 hrs at about 25° C. The resin was washed with DCM and a second lot of Fmoc-Glycine (27 gm, 90 mmol) was dissolved in dichloromethane (250 ml). 1-(2-mesitylene sulfonyl)-3-nitro-1H-1,2,4 triazole (26.6 gm, 90 mmol) and 1-methyl imidazole (5.3 ml, 90 mmol) was then added and stirred for 3 hrs. The resin was washed with DCM and a sample of resin beads were checked for UV analysis. The capping was carried out using acetic anhydride (15 ml) DCM (120 ml) and pyridine (120 ml). The resin was washed with dichloromethane and DMF. The Fmoc protecting group was removed by treatment with 20% piperidine in DMF. The resin was washed repeatedly with DMF. The next amino acid Fmoc-Arg(pbf)-OH (52 gm, 80 mmol) dissolved in 250 ml DMF was then added. The coupling was carried out by addition of HOBt (10.8 gm, 80 mmol) and DIC (6.2 ml, 80 mmol) in DMF. The completion of the coupling was confirmed by a ninhydrin test. After washing the resin, the Fmoc protecting group was removed with 20% piperidine in DMF. These steps were repeated each time with the respective amino acid according to the peptide sequence. After coupling 12^{th} amino acid Fmoc-Lys (Alloc)-OH, deprotection of alloc group is carried out with palladium tetrakis and phenyl silane in DCM. The resin was washed repeatedly with DMF. The next amino acid H-Glu(OH)-NH(palmitoyl)-Otbu (9.9 gm, 0.023 moles) dissolved in 250 ml DMF was then added. The coupling was carried out by addition of HOBt (10.8 gm, 80 mmol) and DIC (6.2 ml, 80 mmol) in DMF. The completion of the coupling was confirmed by a ninhydrin test. After washing the resin, the Fmoc protecting group of Lys was removed with 20% piperidine in DMF. The next amino acid Fmoc-Ala-OH (52 gm, 80 mmol) dissolved in 250 ml DMF was then added. The coupling was carried out by addition of HOBt (10.8 gm, 80 mmol) and DIC (6.2 ml, 80 mmol) in DMF. The completion of the coupling was confirmed by a ninhydrin test. After washing the resin, the Fmoc protecting group was removed with 20% piperidine in DMF. These steps were repeated each time with the respective amino acid according to the peptide sequence. The resin was washed repeatedly with DMF, Methanol and MTBE and dried under vacuum.

Stage II: Cleavage of Liraglutide from Resin Along with Global Deprotection

45 gms of resin obtained in stage I was treated with cleavage cocktail mixture of TFA (462.5 ml), TIPS (12.5 ml), Water (12.5 ml), and Phenol (12.5 ml), stirred at 0° C. for 30 min. and at 25° C. for 3 hrs at 200 RPM. Then the reaction mixture was filtered, repeatedly wash the resin with TFA and the filtrate was concentrated on Rotary evaporator at 30° C. Pour the concentrated solution to MTBE (2 L) at 4° C. slowly and stir for 1 hr. The precipitate obtained is filtered and dried in a vacuum tray drier to afford 18 gm of Liraglutide crude with a purity of 27.5%.

### Stage III: Purification of Crude Liraglutide Using RP HPLC.

The crude Liraglutide (4 gm) of purity around 27.5% is dissolved in 10 mM Tris buffer (120 ml) of pH: 8.00 and 0.5 N NaOH is further added drop wise to the solution for making the crude solid completely dissolved. The solution is further passed through 0.2 micron filter. The Reverse phase C 18—150 Angstrom media (C18 silica media—10 micron particle size) is equilibrated with 10 mM Tris buffer of pH: 8.0 The crude solution is loaded onto the column and the gradient elution is performed as per the below tabular column against the mobile phase B (Acetonitrile).

TABLE 1
Gradient program for pre purification

| Mobile phase A | Mobile phase B | |
|---|---|---|
| (10 mM Tris Buffer) | (Acetonitrile) | Time |
| 60 | 40 | 30 |
| 55 | 45 | 30 |
| 52 | 48 | 30 |
| 51 | 49 | 60 |

The desired fractions are collected in the gradient range of and the fractions (F1, F2, F3, F4 and F5) whose purity >80% are pooled. The pooled fractions are then subjected to further purification.

The Pooled fractions having purity >80% are then subjected to C18 RPHPLC silica media (5 micron particle size) for further purification. The pooled fractions—Feed is diluted with purified water in the ratio of 1:2 (one part of pooled fraction to two parts of purified water) as a part of sample preparation before loading into the column. The media C18 is first equilibrated with 0.1% TFA for 3 column volumes (1 CV=bed volume of media). After equilibration, the sample is loaded onto the column and the gradient elution is performed as per the below tabular column against the mobile phase B (Acetonitrile).

TABLE 2
Gradient program for second purification

| Mobile phase A | Mobile phase B | |
|---|---|---|
| (0.1% TFA) | (Acetonitrile) | Time |
| 70 | 30 | 30 |
| 50 | 50 | 30 |
| 55 | 45 | 40 |
| 58 | 42 | 40 |

The desired fractions are collected in the gradient range of and the fraction whose purity >96% are pooled together and lyophilized to afford 220 mg of Liraglutide trifluoro acetate salt. The pooled fractions and their purity by HPLC are listed in the below table.

| Fraction NO. | HPLC Purity |
|---|---|
| F1 | 95.55% |
| F3 | 98.42% |
| F7 | 97.03% |
| F10 | 95.00% |

The pooled fractions with the purity of average 97% are subjected further to de solvation to remove the Acetonitrile content by Rota vapor. The final solution was filtered through 0.2 micron filter and lyophilized to get Liraglutide API.

### Example 2

Stage I: Preparation of Tentagel SPHB resin-Gly-Arg(pbf)-Gly-Arg(pbf)-Val-Leu-Trp(Boc)-Ala-Ileu-Phe-Glu(Otbu)-Lys-{Glu(OH)-NH(palmitoyl)}-Ala-Ala-Gln(trt)-Gly-OH-Glu(Otbu)-Leu-Tyr(Otbu)-Ser(Otbu)-Ser(Otbu)-Val-Asp(Otbu)-Ser(Otbu)-Thr(Otbu)-Phe-Thr(Otbu)-Gly-Glu(Otbu)-Ala-Boc-His(trt)-OH using Fragment approach.

Fragments used are as follows

### 1. Fmoc-Arg(pbf)-Gly-OH.

### 2. Fmoc-Leu-Ala-Arg(pbf)-OH.

### 3. Fmoc-Ile-Ala-Trp(boc)-OH.

### 4. Fmoc-Glu(Otbu)-Phe-OH.

### 5. Fmoc-Glu(Otbu)-Phe-OH.

6. Fmoc-Lys-Glu-Palmitic acid.

### 7. Fmoc-Gly-Gln(trt)-Ala-Ala-OH.

### 8. Fmoc-Tyr(Otbu)-Leu-Glu(Otbu)-OH.

### 9. Fmoc-Val-Ser(Otbu)-Ser(Otbu)-OH.

### 10. Fmoc-Phe-Thr(Otbu)-Ser(Otbu)-Asp(Otbu)-OH

### 11. Fmoc-Gly-Thr(Otbu)-OH.

### 12. Boc-His(Trt)-Ala-Glu(Otbu)-OH.

Tentagel SPHB resin (30 gm) is swelled in DCM (300 ml) for 1 hr in a sintered flask. DCM was filtered using Vacuum. Fmoc-Glycine (13.8 gm, 46.8 moles) was dissolved in dichloromethane (150 ml). 1-(2-mesitylene sulfonyl)-3-nitro-1H-1,2,4 triazole (13.8 gm, 46.8 moles) and 1-methyl imidazole (2.4 ml, 29.25 moles) was then added. The resulting solution was added to tentagel resin and stirred for 2 hrs at about 25° C. The resin was washed with DCM and a second lot of Fmoc-Glycine (13.8 gm, 46.8 moles) was dissolved in dichloromethane (150 ml). 1-(2-mesitylene sulfonyl)-3-nitro-1H-1,2,4 triazole (13.8 gm, 46.8 moles) and 1-methyl imidazole (2.4 ml, 29.25 moles) was then added and stirred for 2 hrs. The resin was washed with DCM and a sample of resin beads were checked for UV analysis. The Fmoc protecting group was removed by treatment with 20% piperidine in DMF. The resin was washed repeatedly with DMF. The next amino acid fragment 1 Fmoc-Gly-Arg(pbf)-OH (8.25 gm, 11.7 moles) dissolved in 150 ml DMF was then added. The coupling was carried out by addition of HOBt (2.1 gm, 11.7 moles) and DIC (2.5 ml, 11.7 moles) in DMF for 2 hrs. The completion of the coupling was confirmed by a ninhydrin test. After washing the resin, the Fmoc protecting group was removed with 20% piperidine in DMF. These steps were repeated each time with the respective amino acid fragments according to the peptide sequence. The resin was washed repeatedly with DMF, Methanol and MTBE and dried under vacuum.

Stage II: Cleavage of Liraglutide from Resin Along with Global Deprotection

58 gms of resin obtained from stage I was treated with cleavage cocktail mixture of TFA (555 ml), TIPS (15 ml), Water (15 ml), and Phenol (15 ml) and stirred at 0° C. for 30 min. at 25° C. for 3 hrs at 200 RPM. Then filter the reaction mixture, repeatedly wash the resin with TFA and concentrate on Rotary evaporator at 30° C. Pour the concentrated solution to MTBE at 4° C. slowly and stirred for 1 hr. The precipitate obtained was filtered and dried in a vacuum tray drier to afford 23.12 gm of crude Liraglutide with a purity of 36.89%.

### Stage III: Purification of Crude Liraglutide Using RP HPLC.

The crude Liraglutide (4 gm) of purity around 27.5% is dissolved in 10 mM Tris buffer (120 ml) of pH: 8.00 and 0.5 N NaOH is further added drop wise to the solution for making the crude solid completely dissolved. The solution is further passed through 0.2 micron filter. The Reverse phase C 18—150 Angstrom media (Irregular C18 silica media—10 micron particle size) is equilibrated with 10 mM Tris buffer of pH: 8.0 The crude solution is loaded onto the column and the gradient elution is performed as per the below tabular column against the mobile phase B (Acetonitrile).

TABLE 1
Gradient program for pre purification

| Mobile phase A | Mobile phase B | |
|---|---|---|
| (10 mM Tris Buffer) | (Acetonitrile) | Time |
| 60 | 40 | 30 |
| 55 | 45 | 30 |
| 52 | 48 | 30 |
| 51 | 49 | 60 |

The desired fractions are collected in the gradient range of and the fractions (F1, F2, F3, F4 and F5) whose purity >80% are pooled. The pooled fractions then subjected to further purification.

The Pooled fractions having purity >80% are then subjected to C18 RPHPLC silica media (5 micron particle size) for further purification. The pooled fractions—Feed is diluted with purified water in the ratio of 1:2 (one part of pooled fraction to two parts of purified water) as a part of sample preparation before loading into the column. The media C18 is first equilibrated with 0.1% TFA for 3 column volumes (1 CV=bed volume of media). After equilibration, the sample is loaded onto the column and the gradient elution is performed as per the below tabular column against the mobile phase B (Acetonitrile).

TABLE 2
Gradient program for second purification

| Mobile phase A | Mobile phase B | |
|---|---|---|
| (0.1% TFA) | (Acetonitrile) | Time |
| 70 | 30 | 30 |
| 50 | 50 | 30 |
| 55 | 45 | 40 |
| 58 | 42 | 40 |

The desired fractions are collected in the gradient range and the fraction whose purity >96% are pooled together and Lyophilized to afford 865 mg of Liraglutide trifluoro acetate salt. The pooled fractions and their purity by HPLC are listed in the below table.

| Fraction | HPLC |
|---|---|
| NO. | Purity |
| F2 | 98.91% |
| F3 | 99.24% |
| F6 | 98.97% |
| F9 | 98.58% |
| F13 | 98.79% |
| F16 | 98.01% |
| F17-F19 | 94.16% |
| F20-F22 | 86.10% |

The pooled fractions with the purity of average 97% are subjected further to de solvation to remove the Acetonitrile content by Rota vapor. The final solution was filtered through 0.2 micron filter and lyophilized to get Liraglutide API.

1. A process for the preparation of Liraglutide comprising one or more of the following steps:
a) introducing the spacer N^{α}-Palmitoyl-L-γ-glutamyl-OtBu on side chain NH₂ of Lysine of fragment Fmoc-Lys(Alloc)-Glu(Otbu)-Phe-Ile-Ala-Trp(Boc)-Leu-Val-Arg(Pbf)-Gly-Arg(pbf)-Gly attached to solid support;
b) coupling rest of the amino acid sequentially sequence from alpha amino group of Lysine of the fragment of Liraglutide obtained in step (a) to obtain Liraglutide attached to solid support;
c) cleavage of Liraglutide from the solid support;
d) optionally purifying the resulting Liraglutide.

2. The process of claim 1, wherein Wang resin is employed as the solid phase support.

3. The process of claim 1, wherein N^{α}-Palmitoyl-L-γ-glutamyl-OtBu is coupled with the side chain NH₂ of Lysine in fragment Fmoc-Lys(Alloc)-Glu(Otbu)-Phe-Ile-Ala-Trp(Boc)-Leu-Val-Arg(Pbf)-Gly-Arg(pbf)-Gly in the presence of coupling reagent.

4. The process of claim 1, wherein a cocktail mixture is used for the cleavage of the peptide from resin in step c) which cocktail mixture comprises trifluroacetic acid, thioanisole, phenol, water and triisopropylsilane.

5. The process of claim 1, wherein a cocktail mixture is used for the cleavage of the peptide from resin in step c) which cocktail mixture comprises trifluroacetic acid, phenol, water and triisopropylsilane.

6. The process of claim 1, wherein a solvent is employed in step c) which solvent is dichloromethane.

7. The process of claim 1, wherein purification in step d) is performed by reverse phase high performance liquid chromatography, ion exchange chromatography, affinity chromatography in any combinations thereof.

8. A process for the preparation of Liraglutide, comprising one or more of the following steps:
a) sequential coupling of the fragments Fmoc-Arg(pbf)-Gly-OH, Fmoc-Leu-Ala-Arg(Pbf)-OH, Fmoc-Ile-Ala-Trp(Boc)-OH, Fmoc-Glu(OtBu)-Phe-OH, Fmoc-Glu(OtBu)-Phe-OH, Fmoc-Lys-(Glu-(Palmitic Acid)-OH, Fmoc-Gly-Gln(Trt)-Ala-Ala-OH, Fmoc-Tyr(tBu)-Leu-Glu(OtBu)-OH, Fmoc-Val-SerSer(tBu)-Ser(tBu)-OH, Fmoc-Phe-Thr(tBu)-Ser(tBu)-Asp(tBu)-OH, Fmoc-Gly-Thr(tBu)-OH and Boc-His(Trt)-Ala-Glu(OtBu)-OH on Glycine that is already attached to solid support to obtain Liraglutide attached to solid support;
b) cleavage of Liraglutide from the solid support;
c) optionally purifying the resulting Liraglutide.

9. The process of claim 8, wherein tenatgel resin is employed as the solid phase support.

10. The process of claim 8, wherein a cocktail mixture is used for the cleavage of the peptide from resin in step b) which cocktail mixture comprises trifluroacetic acid, phenol, water and triisopropylsilane, respectively.

11. A process for the preparation of Liraglutide comprising introduction of the spacer N^{α}-Palmitoyl-L-γ-glutamyl-OtBu on side chain NH₂ of Lysine of fragment Fmoc-Lys(Alloc)-Glu(Otbu)-Phe-Ile-Ala-Trp(Boc)-Leu-Val-Arg(Pbf)-Gly-Arg(pbf)-Gly.
